(19) United States
(12) Patent Application Publication
(10) Pub. No.: US 2018/0005031 A1
(43) Pub. Date: Jan. 4, 2018

(54) FINGERPRINT IMAGE DETECTING DEVICE AND METHOD

(71) Applicant: Image Match Design Inc., Zhubei City (TW)

(72) Inventor: Zhieng-Chung CHEN, Hsinchu City (TW)

(21) Appl. No.: 15/637,487

(22) Filed: Jun. 29, 2017

(30) Foreign Application Priority Data
Jun. 30, 2016 (TW) 105120779

(51) Int. Cl. G06K 9/00; A61B 5/1172

(52) U.S. Cl. CPC G06K 9/00563; G06K 9/0012; G06K 9/00087; A61B 5/1172; G06K 9/0004; G06K 9/00013; G06K 9/00053

(57) ABSTRACT

By use of the characteristics that an analog-to-digital converter sends out data sequentially when it converts data of a two-dimensional analog image into pixel data, a fingerprint image detecting device and method generate digital output data having a plurality of rows of data, generate a plurality of one-dimensional datum segments linearly from the digital output data, and determine whether the two-dimensional analog image is a real fingerprint image according to the plurality of one-dimensional datum segments. Thus, the detection of a fingerprint image is implemented by means of one-dimensional calculation instead of two-dimensional calculation, thereby effectively reducing computational load and computational time.

### CROSS-REFERENCE TO RELATED APPLICATIONS

This application claims the priority benefit of Taiwan Application No. 105120779, filed 30 Jun. 2016, the contents of which in its entirety are herein incorporated by reference.

### FIELD OF THE INVENTION

The present invention relates generally to image detection and, more particularly, to a fingerprint image detecting device and method using one-dimensional calculation instead of two-dimensional calculation.

### BACKGROUND OF THE INVENTION

Fingerprint-based personal identification requires having a person to be identified to press his/her finger on a sensing unit, obtaining a two-dimensional analog image of the finger, converting the two-dimensional analog image into a two-dimensional digital datum, such as a two-dimensional pixel datum, and comparing the datum with two-dimensional data stored in a database for identification. However, in the process of identification as described above, marks of fingerprint may remain on the sensing unit after the user's finger leaves the sensing unit. For example, a wet finger may leave a fingerprint mark on the sensing unit, so the two-dimensional analog image obtained from the sensing unit may be a remained fingerprint mark, but not an image of a real fingerprint. In this case, if identification is performed using that two-dimensional analog image, mis-operation of fingerprint-based identification can happen, undermining the overall recognition accuracy. Hence, after the sensing unit obtains a two-dimensional analog image, a fingerprint image detecting device is used to determine whether the obtained two-dimensional analog image is a real fingerprint image. Only after the obtained two-dimensional analog image is verified as a real fingerprint image, fingerprint-based identification is performed, so as to prevent mis-operation.

Conventionally, a fingerprint image detecting device first converts a two-dimensional analog image obtained from a sensing unit into two-dimensional digital data, such as two-dimensional pixel data, and reads a plurality of two-dimensional zones of the two-dimensional pixel data. Then it determines whether the two-dimensional analog image is a real fingerprint image according to the average gray-scale value of the pixels in each of the two-dimensional zones and the difference between the maximum representative value and the minimum representative value in the two-dimensional zone. Taking read two-dimensional zones having a plurality of 8×8 pixels for example, computation has to be performed on all the 8×8 pixels of each of the two-dimensional zones, which includes calculating the average gray-scale value of the 64 pixels, sorting the gray-scale values of the 64 pixels to take out the maximum representative value (e.g. the gray-scale value of the 11th greatest one among the 64 pixels) and the minimum representative value (e.g. the gray-scale value of the 11th smallest one among the 64 pixels), and calculating the difference therebetween. In the process of said two-dimensional calculation, each of the two-dimensional zones has 64 pixel data to compute, so the computational load is high. In addition, reading for the two-dimensional pixel data is continuous in terms of time, and the computational load is high. Only when computation of the present two-dimensional zone has been done, the reading for the next two-dimensional zone can be started, and it is impossible to determine whether the two-dimensional analog image is a real fingerprint image immediately after all the two-dimensional pixel data have been read without an additional storage unit for storing data making the determination whether the two-dimensional analog image is a real fingerprint image possible. Such additional storage unit means increased costs and delayed or detained identification. Moreover, the plurality of two-dimensional zones correspond to different sites in the two-dimensional analog image, so parameters associated to the plurality of two-dimensional zones may be different. This means there are too many parameters to analyze, making adjustment of the fingerprint image detecting device difficult.

Another conventional fingerprint image detecting device converts a two-dimensional analog image obtained from the sensing unit into two-dimensional digital data, such as two-dimensional pixel data, and calculates the sum of the gray-scale values of pixels in a preset zone in the pixel data of the two-dimensional. When the sum of the gray-scale values is greater than a threshold, it is determined that the two-dimensional analog image is a real fingerprint image. While this known method features a low computational load, it makes the determination only based on a local zone (i.e. the preset zone) but not the entire two-dimensional pixel data, so mis-determination tends to happen, making the recognition rate of fingerprint images poor.

Hence, there is a need for a device and a method for this purpose that provides advantages of simple computation, low costs and a high recognition rate of fingerprint images.

### SUMMARY OF THE INVENTION

One objective of the present invention is to provide a device and a method for detection of fingerprint images that feature simple computation, low costs and a high recognition rate of fingerprint images.

Another objective of the present invention is to provide a device and a method for detection of fingerprint images that replace two-dimensional calculation with one-dimensional calculation.

A further objective of the present invention is to provide a device and a method for detection of fingerprint images that have high flexibility.

According to the present invention, a fingerprint image detecting device comprises an analog-to-digital converter receiving a two-dimensional analog image, converting the two-dimensional analog image into pixel data, and sequentially transmitting the pixel data so as to generate digital output data having a plurality of rows of data; a reading unit linearly reading the digital output data so as to generate a plurality of one-dimensional datum segments; and a processing unit determining whether the two-dimensional analog image is a real fingerprint image according to the plurality of one-dimensional datum segments.

According to the present invention, a fingerprint image detecting method comprises receiving a two-dimensional analog image, converting the two-dimensional analog image into pixel data, sequentially transmitting the pixel data to generate digital output data having a plurality of rows of data, linearly reading the digital output data so as to generate a plurality of one-dimensional datum segments, and determining whether the two-dimensional analog image is a real fingerprint image according to the plurality of one-dimensional datum segments.

The present invention uses the characteristics of an analog-to-digital converter that it converts two-dimensional analog image data into pixel data and transmits the data sequentially to replace two-dimensional calculation with one-dimensional calculation for detecting fingerprint images, thereby effectively reducing computational load and computational time, so as to simplify the circuit and save costs.

The present invention effectively reduces computational loads and computational time, so as to determine whether a two-dimensional analog image is a real fingerprint image once the digital output data have been read, without delaying or detaining identification. Additionally, by evenly reading a plurality of one-dimensional datum segments in digital output data, the present invention can be regard as determining whether the two-dimensional analog image is a real fingerprint image according to the entire digital output data, and thus provides good recognition for fingerprint images. Preferably, by adjusting the parameters of the read digital output data, the recognition rate for fingerprint images can be changed, adding its use with flexibility.

### BRIEF DESCRIPTION OF THE DRAWINGS

FIG. 1 is a first embodiment of a fingerprint image detecting device of the present invention;

FIG. 2 illustrates how to generate a plurality of one-dimensional datum segments;

FIG. 3 illustrates how to generate a plurality of one-dimensional datum segments;

FIG. 4 is a second embodiment of the fingerprint image detecting device of the present invention;

FIG. 5 is a first embodiment of a processing unit according to the present invention;

FIG. 6 is a second embodiment of the processing unit;

FIG. 7 shows a plurality of one-dimensional quantitative values sorted into a plurality of groups; and

FIG. 8 is a third embodiment of the processing unit.

### DETAILED DESCRIPTION OF THE INVENTION

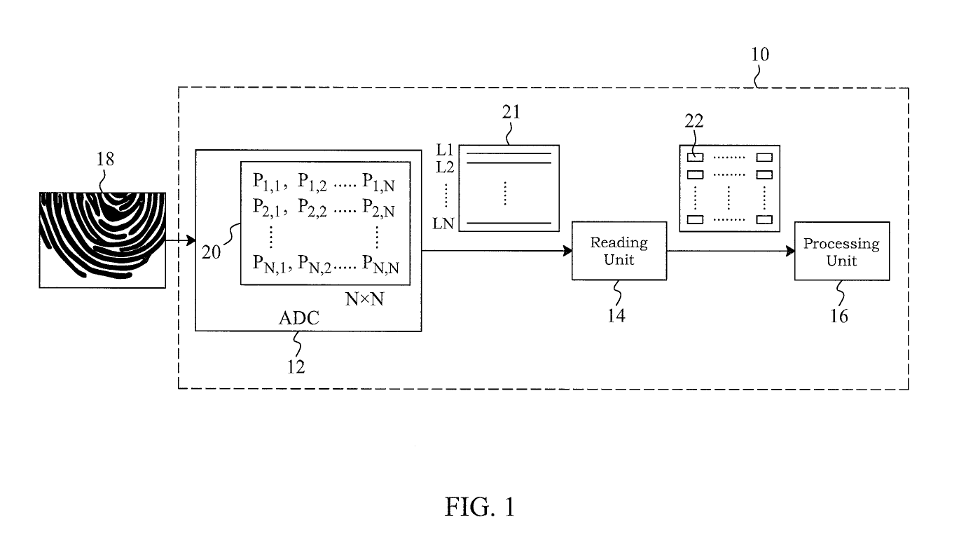

In a first embodiment of the present invention as shown in FIG. 1, a fingerprint image detecting device 10 comprises an analog-to-digital converter (ADC) 12, a reading unit 14, and a processing unit 16. When a two-dimensional analog image 18 enters the fingerprint image detecting device 10, the ADC 12 receives the two-dimensional analog image 18 and converts it into two-dimensional pixel data 20 having N×N quantitative values (e.g. gray-scale values) P_{1, 1}, P_{1, 2} . . . P_{1, N}, P_{2, 1}, P_{2, 2} . . . P_{2, N} . . . P_{N, 1}, P_{N, 2} . . . P_{N, N}, wherein N is a positive integer greater than 1. Since ADC 12 converts the two-dimensional analog image 18 into pixel data 20 and it features transmitting data sequentially, the pixel data 20 are output in rows. The ADC 12 generates digital output data 21 having N rows of data L1, L2 . . . LN, and each row of data L1, L2 . . . LN includes N one-dimensional quantitative values. The reading unit 14 is connected to the ADC 12, and generates a plurality of one-dimensional datum segments 22 linearly from the digital output data 21. Each of the one-dimensional datum segments 22 includes a plurality of one-dimensional quantitative values. For example, each of the one-dimensional datum segments 22 has a plurality of one-dimensional quantitative values in a number not greater than N. The processing unit 16 is connected to the reading unit 14, and determines whether the two-dimensional analog image 18 is a real fingerprint image according to the plurality of one-dimensional datum segments 22. Preferably, for allowing the ADC 12 to receive the image with better clearness, the two-dimensional analog image 18 is first processed for reducing noise, such as having the background filtered out of the two-dimensional analog image 18, and then enters the fingerprint image detecting device 10.

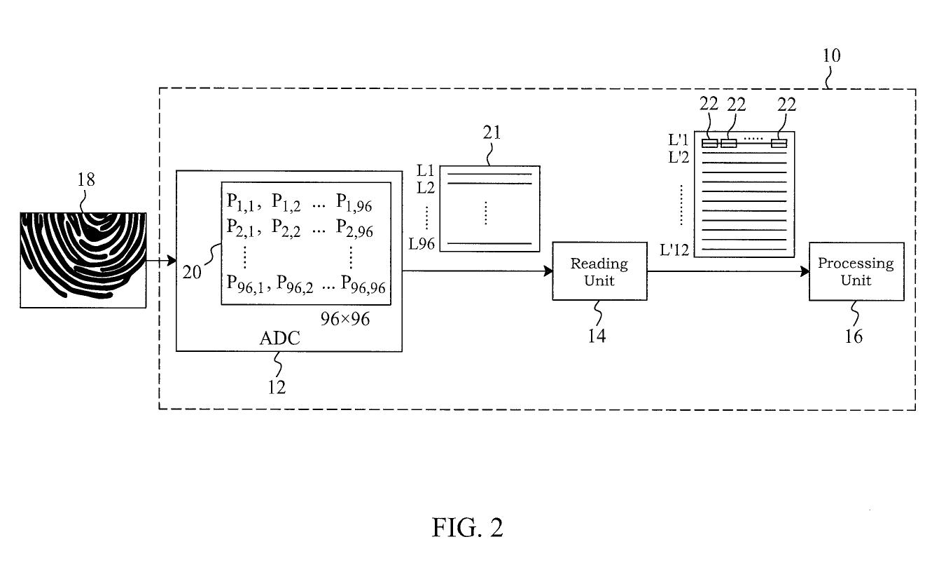

As shown in FIG. 2, in one embodiment, the two-dimensional analog image 18 is converted by the ADC 12 into two-dimensional pixel data 20 having 96×96 quantitative values (e.g. gray-scale values) P_{1, 1}, P_{1, 2} . . . P_{1, 96}, P_{2, 1}, P_{2, 2} . . . P_{2, 96} . . . P_{96, 1}, P_{96, 2} . . . P_{96, 96}. Since the ADC 12 transmits data sequentially, it generates digital output data 21 that have 96 rows of data L1, L2 . . . L96 and each row of data L1, L2 . . . L96 has 96 one-dimensional quantitative values. The reading unit 14 takes out a part of the 96 rows of data in rows, and linearly reads the part of the 96 rows of data in a datum length from the digital output data 21, so as to generate a plurality of one-dimensional datum segments 22. For example, the reading unit 14 takes out a plurality of rows of data from 96 rows of data L1, L2 . . . L96 in rows with a pattern of reading one row every 8, 4, 3 or 2 rows, and reads the plurality of rows of data in a datum length of 6-15 pixels, so as to generate a plurality of one-dimensional datum segments 22. In this embodiment, the reading unit 14 takes out 12 full rows of data L′1, L′2 . . . L′12 from 96 rows of data L1, L2 . . . L96 with the pattern of reading one row every eight (8) rows, and reads the 12 rows of data L′1, L′2 . . . L′12 in the datum length of eight (8) pixels, so as to generate (96÷8)×12=144 one-dimensional datum segment 22. Each of the one-dimensional datum segments 22 includes one-dimensional quantitative values corresponding to the datum length. In other words, each said one-dimensional datum segment 22 includes eight (8) one-dimensional quantitative values. The processing unit 16 determines whether the two-dimensional analog image 18 is a real fingerprint image according to these 144 one-dimensional datum segments 22. Thus, for the digital output data 21 that has 96 rows of data (i.e. L1-L96) each of which has 96 quantitative values, the processing unit 16 only processes 12 rows (i.e. L′1-L′12) of data and performs computation on 144 one-dimensional datum segments 22, without processing or computing any two-dimensional data. In addition, the processing unit 16 when computing each said one-dimensional datum segment 22 only processes eight (8) one-dimensional quantitative values. This not only reduces structural requirements, but also significantly lowers computational loads and computational time. In other word, the present invention uses the characteristic of the ADC 12 that it converts the two-dimensional analog image 18 into pixel data 20 and sequentially transmits the data, and the processing unit 16 determines whether the two-dimensional analog image 18 is a real fingerprint image according to a plurality of one-dimensional datum segments 22, so as to replace two-dimensional calculation with one-dimensional calculation for detecting fingerprint images, thereby significantly reducing computational loads and computational time. In addition, since each said one-dimensional datum segment 22 comprises eight (8) one-dimensional quantitative values, the processing unit 16 only processes eight (8) one-dimensional quantitative values at one time. Thus, once the digital output data 21 necessary for identification have been read, determination of whether the two-dimensional analog image 18 is a real fingerprint image can be done, without delaying or detaining identification. Also the present invention eliminates the need for an additional storage unit that is otherwise required for storing data that verify whether the two-dimensional analog image 18 is a real fingerprint image, thereby being further advantageous for it simplifies circuit and lowers costs.

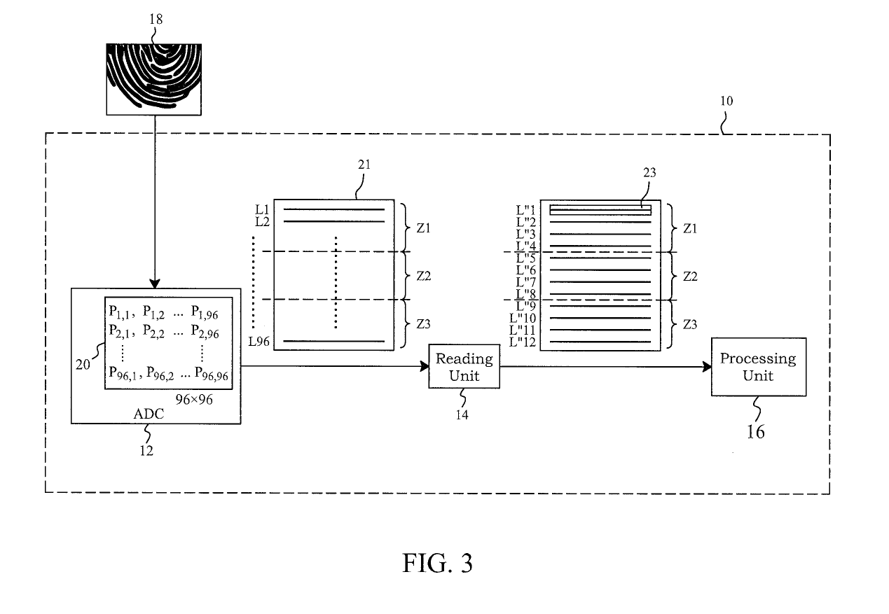

As shown in FIG. 3, in other embodiments, the digital output data 21 have 96 rows of data L1, L2 . . . L96 that are divided into a plurality of zones. The reading unit 14 linearly reads a part of the 96 rows of data L1, L2 . . . L96 in each of the plurality of zones in rows, so as to generate a plurality of one-dimensional datum segments 23, making each of the plurality of zones have a part of the plurality of one-dimensional datum segments 23. For example, the digital output data 21 is divided into 3 zones Z1, Z2 and Z3, and the reading unit 14 reads a part of the 96 rows of data L1, L2 . . . L96 in the zones Z1, Z2 and Z3, respectively, in row, with a pattern of reading one row every 8, 4, 3 or 2 rows, so as to generate a plurality of one-dimensional datum segments 23. In this embodiment, each of the zones Z1, Z2 and Z3 has 32 rows of data of the digital output data 21. The reading unit 14, with a pattern of reading one row every eight (8) rows, reads four (4) rows of data L″1, L″2, L″3, L″4 from the 96 rows of data L1, L2 . . . L96 in the zone Z1 in rows, and reads four (4) rows of data L″5, L″6, L″7, L″8 from the 96 rows of data L1, L2 . . . L96 in the zone Z2 in rows, and reads four (4) rows of data L″9, L″10, L″11, L″12 from the 96 rows of data L1, L2 . . . L96 in the zone Z3 in rows. Each of the rows of data L″1, L″2 . . . L″12 is a one-dimensional datum segment 23, so 12 one-dimensional datum segments 23 are generated. Each of the zones Z1, Z2 and Z3 has a part of the one-dimensional datum segment 23, and each said one-dimensional datum segment 23 comprises 96 one-dimensional quantitative values. The processing unit 16 determines whether the two-dimensional analog image 18 is a real fingerprint image according to these 12 one-dimensional datum segments 23. Thus, for digital output data 21 having 96 rows of data (i.e. L1-L96), the processing unit 16 only processes 12 rows (i.e. L″1-L″12) of one-dimensional data, without processing or computing any two-dimensional data, thereby replacing two-dimensional calculation with one-dimensional calculation for detecting fingerprint images. This effectively lowers structural requirements for hardware and reduces computational loads and computational time, in turn saving costs.

Since the processing unit 16 determines whether the two-dimensional analog image 18 is a real fingerprint image according to the one-dimensional datum segments 22 and 23 generated by the reading unit 14, the recognition rate for fingerprint images is related to the one-dimensional datum segments 22 and 23 generated by the reading unit 14. By adjusting the parameters on which the reading unit 14 reads the digital output data 21, for example the reading pattern and/or the datum length to read, it is possible to adjust the recognition rate for fingerprint images, thereby improving flexibility and convenience in use.

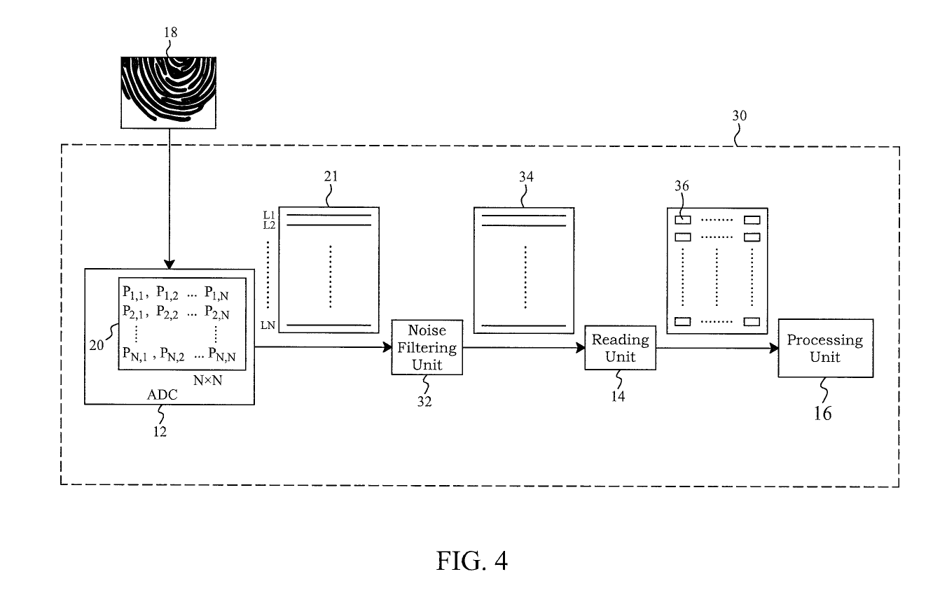

FIG. 4 shows a second embodiment of the present invention. The disclosed fingerprint image detecting device 30 further comprise a noise filtering unit 32 connected between the ADC 12 and the reading unit 14, in addition to the ADC 12, the reading unit 14, and the processing unit 16 as shown in FIG. 1. When the two-dimensional analog image 18 enters the fingerprint image detecting device 30, the ADC 12 receives the two-dimensional analog image 18 and converts the two-dimensional analog image 18 into two-dimensional pixel data 20 having N×N quantitative values (e.g. gray-scale values) P_{1, 1}, P_{1, 2} . . . P_{1, N}, P_{2, 1}, P_{2, 2} . . . P_{2, N} . . . P_{N, 1}, P_{N, 2} . . . P_{N, N}. Since the ADC 12 sequentially transmits the pixel data 20, the ADC 12 generates digital output data 21 having N rows of data L1, L2 . . . LN. Each row of data L1, L2 . . . LN comprises N one-dimensional quantitative values. The noise filtering unit 32 filters noise from the digital output data 21, thereby generating noise-removed digital output data 34 to the reading unit 14. The reading unit 14 linearly generates a plurality of one-dimensional datum segments 36 from the digital output data 34. Each said one-dimensional datum segment 36 comprises a plurality of one-dimensional quantitative values. For example, each said one-dimensional datum segment 36 comprises a plurality of one-dimensional quantitative values in a number that is not greater than N. The processing unit 16 determines whether the two-dimensional analog image 18 is a real fingerprint image according to the plurality of one-dimensional datum segments 36. Therein, the reading unit 14 linearly generates a plurality of one-dimensional datum segments 36 from the digital output data 34 by in the ways as shown in FIG. 2 and FIG. 3, for example. This embodiment uses the noise filtering unit 32 to remove noise from the digital output data 21, so as to make the digital output data 34 provided to the reading unit 14 more reliable. Preferably, the noise filtering unit 32 comprises a low-pass filter for removing high-frequency noise from the digital output data 21.

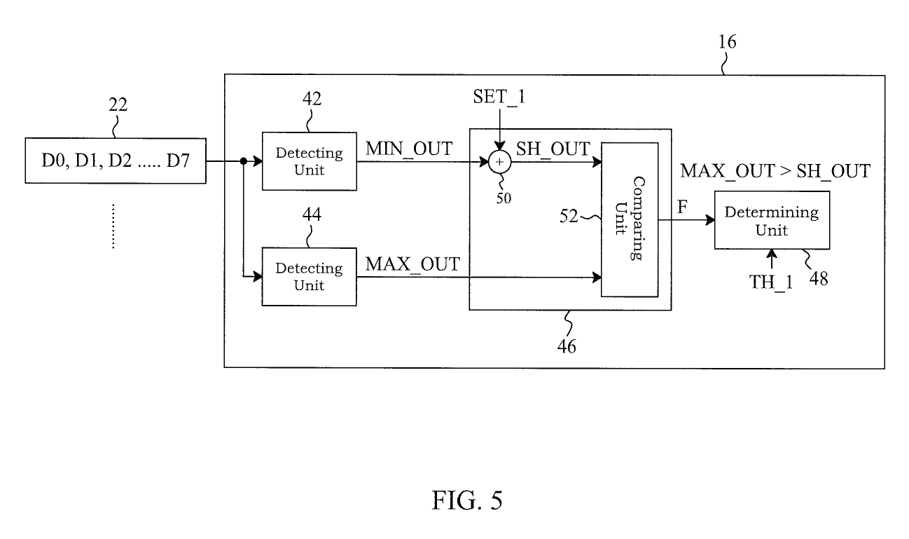

FIG. 5 is a first embodiment of the processing unit 16 as shown in FIG. 1. The processing unit 16 herein comprises detecting units 42 and 44, a flagging unit 46, and a determining unit 48. Referring to FIGS. 1-2 and 5, the reading unit 14 generates a plurality of (e.g. 144) one-dimensional datum segments 22 from the digital output data 21 by means of, for example, the way shown in for example FIG. 2, and sequentially provides them to the processing unit 16. The processing unit 16 deteiiuines whether the two-dimensional analog image 18 is a real fingerprint image according to the plurality of one-dimensional datum segments 22. A real fingerprint image has a fingerprint edge, and there is obvious gray-scale variance at the fingerprint edge. Due to this fact, it is possible to determine whether the two-dimensional analog image 18 is a real fingerprint image by first detecting whether there is obvious gray-scale variance among the plurality of one-dimensional datum segments 22, and learning the proportion or number of the one-dimensional datum segments having a fingerprint edge among the plurality of one-dimensional datum segments 22. When the one-dimensional datum segment 22 having a plurality of (e.g. 8) one-dimensional quantitative values D0, D1, D2 . . . D7 is provided to the processing unit 16, the detecting unit 42 connected to the reading unit 14 selects the minimum representative value MIN_OUT among the one-dimensional quantitative values D0, D1, D2 . . . D7, and the detecting unit 44 connected to the reading unit 14 selects the maximum representative value MAX_OUT among the one-dimensional quantitative values D0, D1, D2 . . . D7. In this embodiment, the minimum one of the one-dimensional quantitative values D0, D1, D2 . . . D7 is selected as the minimum representative value MIN_OUT, and the second greatest one among the one-dimensional quantitative values D0, D1, D2 . . . D7 is selected as the maximum representative value MAX_OUT. For example, where the one-dimensional quantitative values D0-D7 are 0, 70, 200, 150, 120, 60, 40, respectively, the minimum representative value MIN_OUT is 0, and the maximum representative value MAX_OUT is 150. Since the greatest one among the one-dimensional quantitative values D0, D1, D2 . . . D7 may be noise, the second greatest one among the one-dimensional quantitative values is selected as the maximum representative value MAX_OUT, to prevent influence from noise. In other embodiments, the minimum representative value MIN_OUT and the maximum representative value MAX_OUT may be selected according to practical needs. The flagging unit 46 is connected to the detecting units 42 and 44, and serves to compare the minimum representative value MIN_OUT with the maximum representative value MAX_OUT so as to determine whether the one-dimensional datum segment 22 has a fingerprint edge, and generate flag F when the one-dimensional datum segment 22 has a fingerprint edge. For example, when the difference between the maximum representative value MAX_OUT and the minimum representative value MIN_OUT is greater than the preset value SET_1, it means that the one-dimensional datum segment 22 has obvious gray-scale variance, or that the one-dimensional datum segment 22 has a fingerprint edge, so the flagging unit 46 generates a flag F. On the contrary, if the difference between the maximum representative value MAX_OUT and the minimum representative value MIN_OUT is not greater than the preset value SET_1, it means that the one-dimensional datum segment 22 does not have obvious gray-scale variance, or that the one-dimensional datum segment 22 does not have a fingerprint edge, so the flagging unit 46 does not generate a flag F. The preset value SET_1 may be set according to practical needs. For example, when the minimum representative value MIN_OUT and the maximum representative value MAX_OUT in the one-dimensional datum segment 22 are 0 and 150, respectively, the difference between the maximum representative value MAX_OUT and the minimum representative value MIN_OUT is 150. Where the preset value SET_1 is 100, the difference is greater than the preset value SET_1, so the flagging unit 46 generates a flag F. Where the preset value SET_1 is 160, the difference is not greater than the preset value SET_1, so the flagging unit 46 does not generate a flag F. In this embodiment, the flagging unit 46 comprises a shifting unit 50 and a comparing unit 52. The shifting unit 50 is connected to the detecting unit 42, and serves to shift the minimum representative value MIN_OUT for the preset value SET_1, so as to generate a shifted representative value SH_OUT. The comparing unit 52 is connected to shifting unit 50 and the detecting unit 44, and serves to compare the shifted representative value SH_OUT with the maximum representative value MAX_OUT. When the maximum representative value MAX_OUT is greater than the shifted representative value SH_OUT, a flag F is generated. After computation for the present one-dimensional datum segment 22 is done, the detecting units 42 and 44 and the flagging unit 46 then perform computation on the next one-dimensional datum segment 22 in the manner as described above, until all the one-dimensional datum segments 22 (e.g. 144 one-dimensional datum segments 22) have been computed. The determining unit 48 is connected to flagging unit 46, and serves to count the number of the flags F generated in the plurality of one-dimensional datum segments 22, so as to determine whether the two-dimensional analog image 18 is a real fingerprint image. When a ration between the number of the flags F and the total number of the plurality of one-dimensional datum segments 22 is greater than a threshold TH_1, it means that the proportion of the one-dimensional datum segments having a fingerprint edge in all the plurality of (e.g. 144) one-dimensional datum segment 22 is greater than the threshold TH_1 or the number of the one-dimensional datum segments having a fingerprint edge in all the plurality of (e.g. 144) one-dimensional datum segments 22 is greater than a product of the threshold TH_1 and the total number of the plurality of one-dimensional datum segments 22, it is determined that the two-dimensional analog image 18 is a real fingerprint image, wherein the threshold TH_1 may be set according to practical needs. For example, when the total number of the one-dimensional datum segments 22 is 144 and the number of the generated flags is 45, the ratio of the number of the flags F to the total number of the one-dimensional datum segment 22 is 45/144=31.25%. Where the threshold TH_1 is set as 30%, the ratio is greater than threshold TH_1, so it is determined that the two-dimensional analog image 18 is a real fingerprint image. Wherein the threshold TH_1 is set as 35%, the ratio is not greater than the threshold TH_1, so it is determined that the two-dimensional analog image 18 is not a real fingerprint image. This embodiment uses the proportion or number of the one-dimensional datum segments 22 having a fingerprint edge to determine whether the two-dimensional analog image 18 is a real fingerprint image. By adjusting the settings of the preset value SET_1 and the threshold TH_1, the recognition rate for fingerprint images can be improved continuously over time.

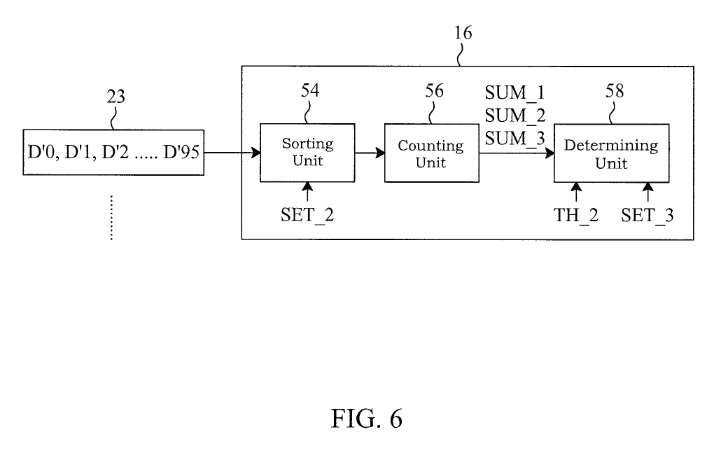

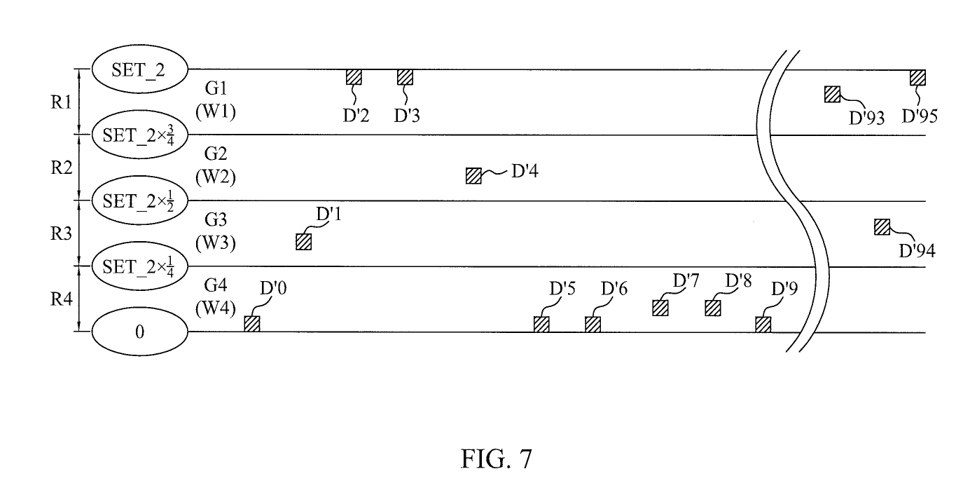

FIG. 6 is a second embodiment of the processing unit 16 as shown in FIG. 1, and the processing unit 16 comprises a sorting unit 54, a counting unit 56, and a determining unit 58. Referring to FIGS. 1, 3 and 6, the digital output data 21 includes a plurality of zones. For example, the digital output data 21 includes three zones Z1, Z2 and Z3. The reading unit 14 provides the processing unit 16 sequentially with a plurality of (e.g. 12) one-dimensional datum segments 23 generated form a plurality of zones (e.g. the zones Z1, Z2 and Z3) by that way as shown in FIG. 3, for example. The processing unit 16 determines whether the two-dimensional analog image 18 is a real fingerprint image according to the plurality of one-dimensional datum segments 23. Since a real fingerprint image has high contrast, it is possible to determine whether the two-dimensional analog image 18 is a real fingerprint image by detecting the number of zones having high contrast among the plurality of zones (e.g. zone Z1, Z2 and Z3). When the one-dimensional datum segment 23 having a plurality of (e.g. 96) one-dimensional quantitative values D′0, D′1, D′2 . . . D′95 is provided to the processing unit 16, the sorting unit 54 connected to reading unit 14 divides the one-dimensional quantitative values D′0, D′1, D′2 . . . D′95 into a plurality of groups according to a preset value SET_2 wherein the groups correspond to a plurality of weights, respectively, so that each said one-dimensional quantitative value D′0, D′1, D′2 . . . D′95 has the weight corresponding to its group. As shown in FIG. 7, in one embodiment, the preset value SET_2 is divided into a plurality of ranges that correspond to the plurality of groups, respectively. For example, the preset value SET_2 is divided into four (4) ranges R1, R2, R3, R4, corresponding to four (4) groups G1, G2, G3, G4, respectively. Particularly, the range R1 is between the preset value SET_2 and three fourths of the preset value SET_2, corresponding to the group G1. The range R2 is between three fourths of the preset value SET_2 and one half of the preset value SET_2, corresponding to the group G2. The range R3 is between one half of the preset value SET_2 and one quarter of the preset value SET_2, corresponding to the group G3. The range R4 is between one quarter of the preset value SET_2 and zero, corresponding to the group G4. The one-dimensional quantitative values D′0, D′1, D′2 . . . D′95 are divided according to the range R1-R4 into the groups G1-G4 that correspond to weights W1-W4, respectively. For example, among one-dimensional quantitative values D′0, D′1, D′2 . . . D′95, those falling within the range R1 are grouped into the group G1, those falling within the range R2 are grouped into the group G2, those falling within the range R3 are grouped into the group G3, and those falling within the range R4 those falling within the group G4. The one-dimensional quantitative values D′0, D′1, D′2 . . . D′95 in the groups G1, G2, G3, G4 each have a weight W1, W2, W3, or W4, where W1>W2>W3>W4. For example, the weights W1, W2, W3, W4 are 4, 2, 1, 0, respectively. The counting unit 56 connected to the sorting unit 54 counts the weights of the one-dimensional quantitative values D′0, D′1, D′2 . . . D′95 in the one-dimensional datum segment 23, i.e. W4+W3+W1+W1+W2+W4+W4+W4+W4+W4+ . . . +W1+W3+W1. When computation for the present one-dimensional datum segment 23 has been done, the sorting unit 54 uses the same way to do computation for the next one-dimensional datum segment 23. The counting unit 56 continues to count the weights corresponding to the one-dimensional quantitative values in the next one-dimensional datum segment 23, until computation for all the one-dimensional datum segments 23 in one zone (e.g. the zone Z1) has been done. At this time, the counting unit 56 generates a count value (e.g. the count value SUM1) that is a count of the weight corresponding to each one-dimensional quantitative value of each said one-dimensional datum segment 23 in the zone (e.g. the zone Z1). After computation for all the one-dimensional datum segments 23 in the zone (e.g. the zone Z1) has been done, the sorting unit 54 and the counting unit 56 perform computation on the one-dimensional datum segments 23 in the next zone in the way as described previously, until computation for the one-dimensional datum segments 23 in all of the zones has been done. For example, the sorting unit 54 and the counting unit 56 then perform computation on the zones Z2 and Z3, so as to generate count values SUM2 and SUM3. The determining unit 58 connected to the counting unit 56 compares the plurality of count values generated by the counting unit 58 with a threshold TH_2, such as comparing the count values SUM1, SUM2, and SUM3 with the threshold TH_2, so as to determine whether the two-dimensional analog image 18 is a real fingerprint image. When some of the count values are greater than the threshold TH_2, it means that the zones having these count values have high contrast. Where the number of the count values in the plurality of count values (e.g. count value SUM1, SUM2, SUM3) is greater than the threshold TH_2 for a preset value SET_3, saying that the number of zones having high contrast (i.e. the zones having the count values greater than the threshold TH_2) in the plurality of zones (e.g. the zones Z1, Z2, Z3) is greater than preset value SET_3, it is determined that the two-dimensional analog image 18 is a real fingerprint image. Therein, the thresholds TH_2 and SET_3 may be set according to practical needs. For example, where the preset value SET_3 is set as 1, when the count values SUM1, SUM2, SUM3 are 38, 40, 45, respectively, if the threshold TH_2 is set as 35, the number of the count values being greater than the threshold TH_2 in the count values SUM1, SUM2, SUM3 is 3, and the number of the zones having high contrast in the zones Z1, Z2, Z3 is 3, being greater than the preset value SET_3, the determining unit 58 determine the two-dimensional analog image 18 is a real fingerprint image. Where the threshold TH_2 is set as 42, the number of the count values being greater than the threshold TH_2 in the count values SUM1, SUM2 is 1, and the number of the zones having high contrast in the zones Z1, Z2, Z3 is 1, being not greater than the preset value SET_3, the determining unit 58 determines that the two-dimensional analog image 18 is not a real fingerprint image. In this embodiment, the preset value SET_2 is the maximum of the two-dimensional analog image 18 converted by the ADC 12. The maximum is related to the amplitude of vibration of the ADC 12. This embodiment determines whether the two-dimensional analog image 18 is a real fingerprint image by detecting how many of the plurality of zones (e.g. zone Z1, Z2, Z3) having high contrast. By adjusting the settings of the preset values SET_2, SET_3 and of the threshold TH_2, it is possible to continuously correct the recognition rate for fingerprint images, so as to improve the recognition rate for fingerprint images over time.

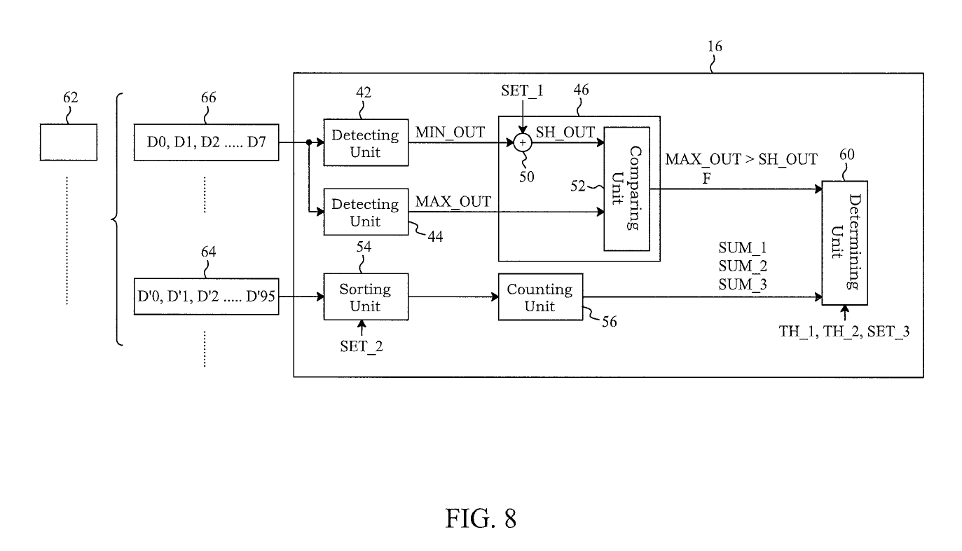

FIG. 8 is a third embodiment of the processing unit 16 as shown in FIG. 1. The processing unit 16 herein comprises a determining unit 60, the detecting units 42 and 44 and the flagging unit 46 as shown in FIG. 5, and the sorting unit 54 and the counting unit 56 as shown in FIG. 6. Referring to FIGS. 1-3 and 8, the processing unit 16 determine whether the two-dimensional analog image 18 is a real fingerprint image according to the plurality of one-dimensional datum segments 62 generated by the reading unit 14. Therein, the plurality of one-dimensional datum segments 62 include a plurality of (e.g. 12) one-dimensional datum segments 64 obtained as shown in FIG. 3 and a plurality of (e.g. 144) one-dimensional datum segments 66 obtained as shown in FIG. 2. Each of the one-dimensional datum segments 64 has a plurality of (e.g. 96) one-dimensional quantitative values (e.g. D′0, D′1, D′2 . . . D′95), and each of the one-dimensional datum segments 66 has a plurality of (e.g. 8) one-dimensional quantitative values (e.g. D0, D1, D2 . . . D7). The plurality of (e.g. 12) one-dimensional datum segments 64 and a plurality of (e.g. 144) one-dimensional datum segments 66 in the plurality of one-dimensional datum segments 62 are respectively provided to the sorting unit 54 and detecting units 42 and 44 that are in the processing unit 16 and connected to the reading unit 14. When the one-dimensional datum segment 66 having a plurality of (e.g. 8) one-dimensional quantitative values (e.g. D0, D1, D2 . . . D7) enters the detecting units 42 and 44, the detecting units 42 and 44 select a minimum representative value MIN_OUT and a maximum representative value MAX_OUT, respectively, from the one-dimensional quantitative values (e.g. D0, D1, D2 . . . D7). The flagging unit 66 connected to the detecting units 42 and 44 compares the minimum representative value MIN_OUT with the maximum representative value MAX_OUT so as to determine whether the one-dimensional datum segment 66 has a fingerprint edge. Where the one-dimensional datum segment 66 has a fingerprint edge, a flag F is generates. For example, the flagging unit 46 generates a flag F when the difference between the maximum representative value MAX_OUT and the minimum representative value MIN_OUT is greater than the preset value SET_1. Therein, the preset value SET_1 may be set according to practical needs. In this embodiment, the flagging unit 46 comprises a shifting unit 50 and a comparing unit 52. The shifting unit 50 shifts the minimum representative value MIN_OUT for the preset value SET_1 so as to generate a shifted representative value SH_OUT. The comparing unit 52 compares the shifted representative value SH_OUT with the maximum representative value MAX_OUT, and generates a flag F when the maximum representative value MAX_OUT is greater than the shifted representative value SH_OUT. The detailed operation of the detecting units 42 and 44 and the flagging unit 46 is as described in FIG. 5, and repeated description is omitted herein. When the one-dimensional datum segment 64 having a plurality of (e.g. 96) one-dimensional quantitative values (e.g. D′0, D′1, D′2 . . . D′95) enters the sorting unit 54, the sorting unit 54 according to a preset value SET_2 divides the one-dimensional quantitative values (e.g. D′0, D′1, D′2 . . . D′95) into a plurality of groups that correspond to a plurality of weights, respectively. The preset value SET_2 is a maximum is obtained as the two-dimensional analog image 18 converted by the ADC 12. The maximum is related to the amplitude of vibration of the ADC 12. The counting unit 56 connected to the sorting unit 54 counts the weights of each said one-dimensional quantitative value (e.g. D′0, D′1, D′2 . . . D′95) of the one-dimensional datum segment 64 in each of the zones (e.g. the zone Z1, zone Z2, zone Z3), so as to generate a plurality of count values (e.g. SUM1, SUM2, SUM3). The detailed operation of the sorting unit 54 and of the counting unit 56 is as shown in FIGS. 6-7, so the repeated description is omitted herein. The determining unit 60 is connected to the flagging unit 46 and the counting unit 56, and serves to count the number of flags F generated in the plurality of one-dimensional datum segment 66 and compare the plurality of count values (e.g. SUM1, SUM2, SUM3) with the threshold TH_2, so as to determine whether the two-dimensional analog image 18 is a real fingerprint image. Where the ratio of the number of the flags F to the total number of the plurality of one-dimensional datum segments 66 is greater than the threshold TH_1 and the number of the count values being greater than the threshold TH_2 in the plurality of count values (e.g. SUM1, SUM2, SUM3) is greater than the preset value SET_3, or the proportion of the datum segments having a fingerprint edge in the plurality of (e.g. 144) one-dimensional datum segments 66 is greater than the threshold TH_1; or where the number of one-dimensional datum segments having a fingerprint edge in the plurality of (e.g. 144) one-dimensional datum segments 66 is greater than the product of the threshold TH_1 and the total number of the plurality of one-dimensional datum segments 66, and the number of zones having high contrast (i.e. the zones having count values greater than the threshold TH_2 in the plurality of zones (e.g. Z1, Z2, Z3) is greater than preset value SET_3, it is determined that the two-dimensional analog image 18 is a real fingerprint image. The preset value SET_3 and the thresholds TH_1, TH_2 may be set according to practical needs. The detailed operation of the determining unit 60 is as described for the determining unit 48 of FIG. 5 and for the determining unit 58 of FIG. 6, so repeated description is omitted herein. This embodiment detects the proportion or number of datum segments having a fingerprint edge in the plurality of one-dimensional datum segment 66, and detects the number of zones having high contrast in the plurality of zones (e.g. the zones Z1, Z2, Z3), thereby determining whether the two-dimensional analog image 18 is a real fingerprint image. This further improves recognition rate for fingerprint images. In addition, by adjusting the settings of the preset values SET_1, SET_2, SET_3 and of the thresholds TH_1, TH_2, it is possible to continuously correct the recognition rate for fingerprint images, so as to improve the recognition rate for fingerprint images over time.

What is claimed is:

1. A fingerprint image detecting device comprising:
an analog-to-digital converter receiving a two-dimensional analog image, and converting the two-dimensional analog image into pixel data and sequentially transmitting the pixel data so as to generate digital output data having a plurality of rows of data;
a reading unit connected to the analog-to-digital converter, generating a plurality of one-dimensional datum segments linearly from the digital output data; and
a processing unit connected to the reading unit, determining whether the two-dimensional analog image is a real fingerprint image according to the plurality of one-dimensional datum segments.

2. The fingerprint image detecting device of claim 1, wherein the pixel data include gray-scale values of the two-dimensional analog image.

3. The fingerprint image detecting device of claim 1, further comprising a noise filtering unit filtering out noise from the digital output data.

4. The fingerprint image detecting device of claim 3, wherein the noise filtering unit comprises a low-pass filter filtering out high-frequency noise from the digital output data.

5. The fingerprint image detecting device of claim 1, wherein the reading unit takes a part of data in rows from the digital output data and reads the taken-out rows of data with a datum length so as to generate the plurality of one-dimensional datum segments, each of the plurality of one-dimensional datum segments including a plurality of one-dimensional quantitative values corresponding to the datum length.

6. The fingerprint image detecting device of claim 5, wherein the processing unit comprises:
a first detecting unit connected to the reading unit, selecting a minimum representative value from the plurality of one-dimensional quantitative values of each of the plurality of one-dimensional datum segments;
a second detecting unit connected to the reading unit, selecting a maximum representative value form the plurality of one-dimensional quantitative values of each of the plurality of one-dimensional datum segments;
a flagging unit connected to the first and second detecting unit, comparing the maximum representative value with the minimum representative value of each of the plurality of one-dimensional datum segments, and generating a flag when a difference between the maximum representative value and the minimum representative value is greater than a preset value; and
a determining unit connected to the flagging unit, counting a number of the generated flags in the plurality of one-dimensional datum segments, and identifying the two-dimensional analog image as a real fingerprint image when a ratio of the number of the flags to a total number of the plurality of one-dimensional datum segments is greater than a threshold.

7. The fingerprint image detecting device of claim 6, wherein the minimum representative value includes a minimum one among the plurality of one-dimensional quantitative values of each of the plurality of one-dimensional datum segments.

8. The fingerprint image detecting device of claim 6, wherein the maximum representative value includes a second greatest one among the plurality of one-dimensional quantitative values of each of the plurality of one-dimensional datum segments.

9. The fingerprint image detecting device of claim 6, wherein the flagging unit comprises:
a shifting unit connected to the first detecting unit, shifting the minimum representative value of each of the plurality of one-dimensional datum segments for the preset value so as to generate a shifted representative value for each of the plurality of one-dimensional datum segments; and
a comparing unit connected to the shifting unit and the second detecting unit, comparing the shifted representative value with the maximum representative value of each of the plurality of one-dimensional datum segments, and generating the flag when the maximum representative value is greater than the shifted representative value.

10. The fingerprint image detecting device of claim 1, wherein the digital output data include a plurality of zones, and the reading unit reads a part of the plurality of rows of data from each of the plurality of zones in rows, so as to generate the plurality of one-dimensional datum segments, each of which includes a plurality of one-dimensional quantitative values.

11. The fingerprint image detecting device of claim 10, wherein the processing unit comprises:
a sorting unit connected to the reading unit, sorting the plurality of one-dimensional quantitative values of the plurality of one-dimensional datum segments in each of the plurality of zones into a plurality of groups corresponding to a plurality of weights respectively, according to a first preset value;
a counting unit connected to the sorting unit, counting the weights corresponding to each of the plurality of one-dimensional quantitative values of the plurality of one-dimensional datum segments in each of the plurality of zones, so as to generate a plurality of count values; and
a determining unit connected to the counting unit, comparing the plurality of count values with a threshold, and identifying the two-dimensional analog image as a real fingerprint image when a number of the plurality of count values that are greater than the threshold is greater than a second preset value.

12. The fingerprint image detecting device of claim 11, wherein the sorting unit divides the first preset value to generate a plurality of ranges corresponding to the plurality of groups respectively, and sorts the plurality of one-dimensional quantitative values of the plurality of one-dimensional datum segments in each of the plurality of zones into the plurality of groups according to the plurality of ranges.

13. The fingerprint image detecting device of claim 1, wherein the digital output data include a plurality of zones, and the reading unit reads a first part of the plurality of rows of data from each of the plurality of zones in rows, so as to obtain a plurality of first one-dimensional datum segments, and takes a second part of the plurality of rows of data from the digital output data in rows and reads the second part of the plurality of rows of data in a datum length, so as to obtain a plurality of second one-dimensional datum segments, thereby generating the plurality of one-dimensional datum segments including the plurality of first one-dimensional datum segments and the plurality of second one-dimensional datum segments, each of the plurality of first one-dimensional datum segments including a plurality of first one-dimensional quantitative values, and each of the plurality of second one-dimensional datum segments including a plurality of second one-dimensional quantitative values corresponding to the datum length.

14. The fingerprint image detecting device of claim 13, wherein the processing unit comprises:
a first detecting unit connected to the reading unit, selecting a minimum representative value among the plurality of second one-dimensional quantitative values of each of the plurality of second one-dimensional datum segment;
a second detecting unit connected to the reading unit, selecting a maximum representative value among the plurality of second one-dimensional quantitative values of each of the plurality of second one-dimensional datum segments;
a flagging unit connected to the first and second detecting units, comparing the maximum representative value with the minimum representative value in each of the plurality of second one-dimensional datum segment, and generating a flag when a difference between the maximum representative value and the minimum representative value is greater than a first preset value;
a sorting unit connected to the reading unit, sorting the plurality of first one-dimensional quantitative values of the plurality of first one-dimensional datum segment in each of the plurality of zones into a plurality of groups corresponding to a plurality of weights respectively, according to a second preset value;
a counting unit connected to the sorting unit, counting weights corresponding to each of the plurality of first one-dimensional quantitative values of the plurality of first one-dimensional datum segments in each of the plurality of zones, so as to generate a plurality of count values; and
a determining unit connected to the flagging unit and the counting unit, counting a number of the flags generated in the plurality of second one-dimensional datum segments, comparing the plurality of count values with the first threshold, and identifying the two-dimensional analog image as a real fingerprint image when a ratio of the number of the flags generated in the plurality of second one-dimensional datum segments to a total number of the plurality of second one-dimensional datum segments is greater than a second threshold and the plurality of count values are greater than the first threshold by more than a third preset value.

15. The fingerprint image detecting device of claim 14, wherein the minimum representative value includes a minimum one among the plurality of second one-dimensional quantitative values of each of the plurality of second one-dimensional datum segments.

16. The fingerprint image detecting device of claim 14, wherein the maximum representative value includes a second greatest one among the plurality of second one-dimensional quantitative values of each of the plurality of second one-dimensional datum segment.

17. The fingerprint image detecting device of claim 14, wherein the flagging unit comprises:
a shifting unit connected to the first detecting unit, shifting the minimum representative value of each of the plurality of second one-dimensional datum segments for the first preset value so as to generate a shifted representative value for each of the plurality of second one-dimensional datum segment; and
a comparing unit connected to the shifting unit and the second detecting unit, comparing the shifted representative value with the maximum representative value of each of the plurality of second one-dimensional datum segment, and generating the flag when the maximum representative value is greater than the shifted representative value.

18. The fingerprint image detecting device of claim 14, wherein the sorting unit divides the second preset value to generate a plurality of ranges corresponding to the plurality of groups respectively, and sorting the plurality of first one-dimensional quantitative values of the plurality of first one-dimensional datum segment in each of the plurality of zones into the plurality of groups according to the plurality of ranges.

19. A fingerprint image detecting method comprising the steps of:
A.) receiving a two-dimensional analog image;
B.) converting the two-dimensional analog image into pixel data and sequentially transmitting the pixel data so as to generate digital output data having a plurality of rows of data;
C.) generating a plurality of one-dimensional datum segments linearly from the digital output data; and
D.) according to the plurality of one-dimensional datum segments determining whether the two-dimensional analog image is a real fingerprint image.

20. The fingerprint image detecting method of claim 19, wherein the step B comprises the step of converting the two-dimensional analog image into gray-scale values.

21. The fingerprint image detecting method of claim 19, further comprising the step of removing noise from the digital output data.

22. The fingerprint image detecting method of claim 19, further comprising the step of removing high-frequency noise from the digital output data.

23. The fingerprint image detecting method of claim 19, wherein the step C comprises the step of taking a part of data in rows from the digital output data and reading the taken-out rows of data with a datum length so as to generate the plurality of one-dimensional datum segments, each of the plurality of one-dimensional datum segments including a plurality of one-dimensional quantitative values corresponding to the datum length.

24. The fingerprint image detecting method of claim 23, wherein the step D comprises the steps of:
E.) selecting a minimum representative value from the plurality of one-dimensional quantitative values of each of the plurality of one-dimensional datum segments;
F.) selecting a maximum representative value from the plurality of one-dimensional quantitative values of each of the plurality of one-dimensional datum segments;
G.) comparing the maximum representative value with the minimum representative value of each of the plurality of one-dimensional datum segments, and generating a flag when a difference between the maximum representative value and the minimum representative value is greater than a preset value; and
H.) counting a number of the flags generated in the plurality of one-dimensional datum segments, and identifying the two-dimensional analog image as a real fingerprint image when a ratio of the number to a total number of the plurality of one-dimensional datum segments is greater than a threshold.

25. The fingerprint image detecting method of claim 24, wherein the step E comprises the step of selecting a minimum one among the plurality of one-dimensional quantitative values of each of the plurality of one-dimensional datum segments as the minimum representative value.

26. The fingerprint image detecting method of claim 24, wherein the step F comprises the step of selecting a second greatest among the plurality of one-dimensional quantitative values of each of the plurality of one-dimensional datum segments as the maximum representative value.

27. The fingerprint image detecting method of claim 24, wherein the step G comprises the steps of:
shifting the minimum representative value of each of the plurality of one-dimensional datum segments for the preset value so as to generate a shifted representative value for each of the plurality of one-dimensional datum segments; and
comparing the shifted representative value with the maximum representative value of each of the plurality of one-dimensional datum segments, and generating the flag when the maximum representative value is greater than the shifted representative value.

28. The fingerprint image detecting method of claim 19, wherein the step C comprises the steps of:
dividing the digital output data to generate a plurality of zones; and
reading a part of the plurality of rows of data from each of the plurality of zones in rows, so as to generate the plurality of one-dimensional datum segments, each of which includes a plurality of one-dimensional quantitative values.

29. The fingerprint image detecting method of claim 28, wherein the step D comprises the steps of:
E.) according to a first preset value, sorting the plurality of one-dimensional quantitative values of the plurality of one-dimensional datum segments in each of the plurality of zones into a plurality of groups corresponding to a plurality of weights respectively;
F.) counting the weights corresponding to each of the plurality of one-dimensional quantitative values of the plurality of one-dimensional datum segments in each of the plurality of zones, so as to generate a plurality of count values; and
G.) comparing the plurality of count values with a threshold, and identifying the two-dimensional analog image as a real fingerprint image when a number of the plurality of count values that are greater than the threshold is greater than a second preset value.

30. The fingerprint image detecting method of claim 29, wherein the step E comprises the steps of:
dividing the first preset value to generate a plurality of ranges corresponding to the plurality of groups respectively; and
according to the plurality of ranges, sorting the plurality of one-dimensional quantitative values of the plurality of one-dimensional datum segments in each of the plurality of zones into the plurality of groups.

31. The fingerprint image detecting method of claim 19, wherein the step C comprises the steps of:
dividing the digital output data to generate a plurality of zones;
reading a first part of the plurality of rows of data from each of the plurality of zones in rows, so as to obtain a plurality of first one-dimensional datum segments, each of which includes a plurality of first one-dimensional quantitative values;
taking a second part of the plurality of rows of data from the digital output data in rows and reading the second part of the plurality of rows of data in a datum length, so as to obtain a plurality of second one-dimensional datum segments, each of which includes a plurality of second one-dimensional quantitative values corresponding to the datum length; and
generating the plurality of one-dimensional datum segments including the plurality of first one-dimensional datum segment and the plurality of second one-dimensional datum segment.

32. The fingerprint image detecting method of claim 31, wherein the step D comprises the steps of:
E.) selecting a minimum representative value among the plurality of second one-dimensional quantitative values of each of the plurality of second one-dimensional datum segment;
F.) selecting a maximum representative value among the plurality of second one-dimensional quantitative values of each of the plurality of second one-dimensional datum segment;
G.) comparing the maximum representative value with the minimum representative value of each of the plurality of second one-dimensional datum segment, and generating a flag when a difference between the maximum representative value and the minimum representative value is greater than a first preset value;
H.) according to a second preset value, sorting the plurality of first one-dimensional quantitative values of the plurality of first one-dimensional datum segment in each of the plurality of zones into a plurality of groups corresponding to a plurality of weights respectively;
I.) counting weights corresponding to each of the plurality of first one-dimensional quantitative values of each of the plurality of first one-dimensional datum segment in the plurality of zones, so as to generate a plurality of count values; and
J.) counting a number of the flags generated in the plurality of second one-dimensional datum segments, comparing the plurality of count values with a first threshold, and identifying the two-dimensional analog image as a real fingerprint image when a ratio of the number of the flags generated in the plurality of second one-dimensional datum segments to a total number of the plurality of second one-dimensional datum segments is greater than a second threshold and the plurality of count values are greater than the first threshold by more than a third preset value.

33. The fingerprint image detecting method of claim 32, wherein the step E comprises the step of selecting a minimum one among the plurality of second one-dimensional quantitative values of each of the plurality of second one-dimensional datum segment as the minimum representative value.

34. The fingerprint image detecting method of claim 32, wherein the step F comprises the step of selecting a second greatest one among the plurality of second one-dimensional quantitative value of each of the plurality of second one-dimensional datum segment as the maximum representative value.

35. The fingerprint image detecting method of claim 32, wherein the step G comprises the steps of:
shifting the minimum representative value of each of the plurality of second one-dimensional datum segment for the first preset value so as to generate a shifted representative value for each of the plurality of second one-dimensional datum segment; and
comparing the shifted representative value with the maximum representative value of each of the plurality of second one-dimensional datum segment, and generating the flag when the maximum representative value is greater than the shifted representative value.

36. The fingerprint image detecting method of claim 32, wherein the step H comprises the steps of:
dividing the second preset value to generate a plurality of ranges corresponding to the plurality of groups respectively; and
according to the plurality of ranges, sorting the plurality of first one-dimensional quantitative values of the plurality of first one-dimensional datum segment in each of the plurality of zones into the plurality of groups.
